(19) United States
(12) Patent Application Publication
(10) Pub. No.: US 2008/0293597 A1
(43) Pub. Date: Nov. 27, 2008

(54) METHOD FOR INHIBITING HYDRATE FORMATION

(75) Inventor: Gordon T. Rivers, Houston, TX (US)

Correspondence Address: MADAN, MOSSMAN & SRIRAM, P.C., 2603 AUGUSTA DRIVE, SUITE 700, HOUSTON, TX, 77057-5662, US

(73) Assignee: BAKER HUGHES INCORPORATED, Houston, TX (US)

(21) Appl. No.: 12/184,912

(22) Filed: Aug. 1, 2008

Related U.S. Application Data
(63) Continuation-in-part of application No. 11/485,780, filed on Jul. 13, 2006.

(51) Int. Cl. C09K 8/528; C07C 211/62

(52) U.S. Cl. 507/240; 564/281

(57) ABSTRACT

A sterically hindered quaternary ammonium composition is prepared by contacting a solvent having hydroxyl functionality, a halohydrin, and a sterically hindered tertiary amine, under reaction conditions sufficient to produce a sterically hindered quaternary ammonium compound. The reaction proceeds with excellent yield. The resulting compounds are particularly useful for inhibiting formation of hydrates in hydrocarbon reservoirs and pipelines. Novel compositions of matter include sterically hindered quaternary ammonium compounds conforming to the formulas C₂₇H₅₈XNO₂ and C₂₉H₆₃XNO₂, wherein X is a halogen.

### CROSS REFERENCE TO RELATED APPLICATIONS

This application is a Continuation-in-Part of U.S. patent application Ser. No. 11/485,780 which was filed on Jul. 13, 2006, and which is fully incorporated herein by reference.

### BACKGROUND OF THE INVENTION

1. Field of the Invention

This invention relates to quaternary ammonium compounds. More particularly, this invention relates to sterically hindered quaternary ammonium compounds used as hydrate inhibitors in the oilfield industry.

2. Background Art

In the oilfield drilling industry a problem is often encountered with formation of hydrocarbon hydrates. A number of hydrocarbons, especially lower-boiling light hydrocarbons such as methane that are frequently included in formation fluids and natural gas deposits, tend to form hydrates in conjunction with water. Even non-hydrocarbons, such as carbon dioxide, nitrogen and hydrogen sulfide, may form hydrates under certain conditions. Hydrates of all kinds are encouraged by combinations of lower temperatures and higher pressures, such as are encountered in subsea projects. The hydrates thus formed usually exist as solids that are highly insoluble in the formation fluids and natural gases in which they are found. Because of this insolubility, the hydrocarbon hydrates create problems during production and transport of the fluids, because they may cause clogging of flowlines, pipelines, other types of transfer lines and conduits, and of valves and safety devices. Such clogging may result in shutdowns, losses of production, risks of explosion, and unintended releases of hydrocarbons into the environment.

A number of methods for inhibiting formation of these hydrocarbon hydrates have been developed. One known to be relatively effective involves the addition of quaternary ammonium compounds. The quaternary ammonium compounds are hypothesized to kinetically inhibit formation of the clathrate cage or lattice structure of the hydrate. This may occur when the nitrogen atom of the quaternary ammonium compound replaces an oxygen atom in the “host” water molecule, followed by replacement of other hydrocarbon “guest” molecules by the quaternary ammonium compound's substituent groups. Once these replacements have occurred, the further growth and agglomeration of the cage or lattice structures are effectively prevented or discouraged. Unfortunately, not all quaternary ammonium compounds are equally effective at such inhibition, since a number of factors may determine the speed and relative number of replacements accomplished. Furthermore, quaternary ammonium compounds in general tend to be relatively expensive to produce, and many methods for producing them suffer from relatively low yields.

In view of the above, it would be desirable in the art to find quaternary ammonium compounds that are effective at inhibiting hydrate formation, particularly for oilfield purposes, and that are relatively less expensive to produce or use.

### SUMMARY OF THE INVENTION

Accordingly, it has been found that certain specifically sterically hindered quaternary ammonium compounds can be produced by a method comprising contacting a halohydrin and a sterically hindered tertiary amine in a solvent having hydroxyl functionality, under reaction conditions such that a sterically hindered quaternary ammonium compound is produced.

These compounds have been found to be useful in applications for treating fluid passing through oil and natural gas exploration and or production equipment to eliminate or mitigate the formation of hydrates. A method of employing these compounds includes detecting a reduction of flow rate for fluid passing through a wellbore, flowline, pipeline, transfer line, or tubing; determining whether temperature and/or pressure within the wellbore, flowline, pipeline, transfer line, or tubing are sufficient for hydrate formation; and if the temperature and/or pressure within the wellbore, flowline, pipeline, transfer line, or tubing are sufficient for hydrate formation, then contacting the fluid within the wellbore, flowline, pipeline, transfer line, or tubing.

The invention also includes, in one aspect, a method of inhibiting hydrate formation in a fluid, comprising contacting a halohydrin and a sterically hindered tertiary amine and a solvent having hydroxyl functionality, under reaction conditions such that a sterically hindered quaternary ammonium compound is produced, and contacting the sterically hindered quaternary ammonium compound and a fluid comprising water and a compound selected from the group consisting of a hydrocarbon, carbon dioxide, nitrogen, hydrogen sulfide, and mixtures thereof, such that the formation of hydrates in the fluid is inhibited in comparison with a similar fluid without the sterically hindered quaternary ammonium compound.

The present invention also includes a sterically hindered quaternary ammonium composition conforming to the general formula

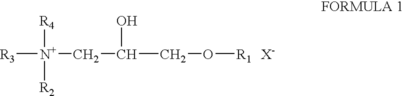

wherein R₁ has 12 or 14 carbon atoms; wherein R₂, R₃ and R₄ are each independently butyl or isobutyl moieties, may be linear or branched, and may have one or more carbon-to-carbon double bonds; and wherein X is a halogen selected from the group consisting of chlorine, fluorine, bromine and iodine.

The present invention also includes a method of inhibiting hydrate formation comprising contacting a fluid comprising water and a compound selected from the group consisting of a hydrocarbon, carbon dioxide, nitrogen, hydrogen sulfide, and mixtures thereof, with a sterically hindered quaternary ammonium composition conforming to the general formula

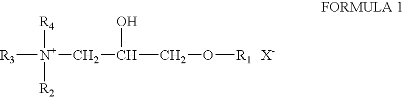

wherein R₁ has from 1 to 20 carbon atoms; wherein R₂, R₃ and R₄ each independently has from 3 to 6 carbon atoms, may be linear or branched, and may have one or more carbon-to-carbon double bonds; and wherein X is a halogen selected from the group consisting of chlorine, fluorine, bromine and iodine; under reaction conditions such that the formation of hydrates in the fluid is inhibited in comparison with a similar fluid without the sterically hindered quaternary ammonium compound.

The present invention further includes a method of inhibiting the formation of hydrates comprising contacting a fluid comprising water and a compound selected from the group consisting of a hydrocarbon, carbon dioxide, nitrogen, hydrogen sulfide, and mixtures thereof, with a sterically hindered quaternary ammonium composition conforming to the formula

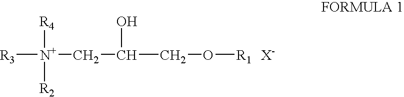

wherein R₁ has 12 or 14 carbon atoms; wherein R₂, R₃ and R₄ are each independently butyl or isobutyl moieties, may be linear or branched, and may have one or more carbon-to-carbon double bonds; and wherein X is a halogen selected from the group consisting of chlorine, fluorine, bromine and iodine; under conditions such that the formation of hydrates in the fluid is inhibited in comparison with a similar fluid without the sterically hindered quaternary ammonium compound.

### DETAILED DESCRIPTION OF THE INVENTION

The present invention may allow a variety of quaternary ammonium compounds to be prepared in good yield without olefin formation occurring and with excellent conversion. These compounds show significant steric hindrance, which may serve to inhibit formation of hydrates that include, for example, hydrocarbons having a variety of carbon chain lengths. The compounds may further serve the same purpose with respect to materials that may form non-hydrocarbon hydrates, such as carbon dioxide, nitrogen and hydrogen sulfide.

While it is hypothesized that the reaction to prepare the sterically hindered quaternary ammonium compounds occurs in at least three discrete events, it is emphasized that the method may be carried out as a single step, thereby simplifying and facilitating production. Without wishing to be bound by any mechanism or theory thereof, it is hypothesized that these events include, first, the amine acting as a base which effects epoxidation of the halohydrin and also forms an amine hydrohalide. Second, the amine hydrohalide salt exists in equilibrium with the protonated epoxide and the amine. Third, the protonated epoxide reacts with the amine to form the quaternary ammonium compound. It is further hypothesized that all three reactions are favored by the hydroxyl functionality of the solvent.

The starting materials for the inventive method include, first, the halohydrin. This compound is defined herein as a type of chemical compound or functional group in which one carbon atom has a substituent of the halogen group in a carbon-carbon saturated covalent bond, and the other carbon atom has a hydroxyl substituent. These compounds adhere to the general formula

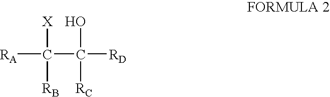

wherein X is chlorine, fluorine, bromine or iodine; and wherein R_{A}, R_{B}, R_{C} and R_{D} are independently selected from hydrogen, hydrocarbon substituents containing from 1 to 20 carbon atoms, and heteroatoms selected from oxygen, nitrogen, phosphorus and combinations thereof.

Thus, simple halohydrins would include, for example, 1-halo-2-hydroxyl ethane (X—CH₂CH₂—OH), and 1-halo-2,3-hydroxylpropane (X—CH₂—CH₂CH₂OH), wherein X is the halogen. Halohydrins may be, in general, formed from an alkene in a halohydrin formation reaction, or from an epoxide by reaction with a hydrohalic acid. Those skilled in the art will be aware of means and methods to prepare these materials without further direction herein, and alternatively such materials may be, in some instances, purchased commercially.

The second starting material is the solvent having hydroxyl functionality. This solvent may be selected from the group consisting of water; alcohols containing from 1 to 10 carbon atoms; glycols; and mixtures thereof. Of these, methanol, ethanol, water, and mixtures thereof may, in some non-limiting embodiments, be particularly preferred for reasons of economy and yield. In other non-limiting embodiments, glycols, longer-chain alcohols, or mixtures of any of these with one another or with water, methanol, and/or ethanol, may be effectively employed. Among suitable and commercially available glycols are, for example, ethylene glycol and propylene glycol.

The third starting material is a sterically hindered tertiary amine. This tertiary amine is, in some non-limiting embodiments, a tertiary amine conforming to the general formula

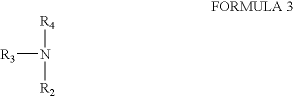

wherein R₂, R₃ and R₄ each independently have from 3 to 12 carbon atoms, may be linear or branched, and may have one or more carbon-to-carbon double bonds. In some non-limiting embodiments the R₂, R₃ and R₄ groups each independently have from 3 to 5 carbon atoms. Amines having 3 carbon atoms for each group may be selected from the group consisting of tripropylamine and triallylamine (also called triprop-2-enylamine). Amines having 4 carbon atoms for each group include tributylamine, triisobutylamine (i.e., tri-2-methylpropylamine), trimethyl-allylamine (i.e., tri-2-methylprop-2-enylamine), tribut-2-enylamine, and tribut-3-enylamine. Amines having 5 carbon atoms for each group include tripentylamine, triisopentylamine (i.e., tri-3-methylbutylamine), tri-3-methylbut-2-enylamine, and tri-3-methylbut-3-enylamine. Mixtures of any of the above may also be employed. In some non-limiting embodiments the sterically hindered tertiary amine may be selected from the group consisting of tripropylamine, tributylamine, triisobutylamine, and mixtures thereof. In other non-limiting embodiments, tributylamine and triisobutylamine are selected, and in still other non-limiting embodiments, tributylamine is selected.

The proportions of the starting materials may be conveniently calculated based on the molar ratio of the halohydrin to the amine. While a wide range of ratios may be employed, it will be obvious to those skilled in the art that controlling the molar ratios within certain ranges will help to optimize conversions, i.e., yields. In one non-limiting embodiment the mole ratio of halohydrin to amine may be from about 2:1 to about 1:2. In another non-limiting embodiment a mole ratio of from about 1.5:1 to about 1:1.5 may be employed. In yet another non-limiting embodiment, a ratio of from about 1.15:1 to about 1:1.15 may be employed.

Once a mole ratio of halohydrin to amine is selected, the amount of hydroxyl functionality containing solvent may be estimated as a weight percentage of the total mass contained in the reaction vessel. The solvent may, in some non-limiting embodiments, range from about 1 to about 99 percent by weight. In other non-limiting embodiments it may be in the range of from about 2 to about 80 percent by weight. In certain particularly desirable but non-limiting embodiments, a solvent in the range of from about 5 to about 50 percent by weight may result in a product that is not excessively dilute and yet still exhibits a relatively high rate of conversion to the quaternary ammonium compound, within a reasonable time.

The reaction conditions are, in some non-limiting embodiments, based upon a combination of temperature and pressure sufficient to yield the sterically hindered quaternary ammonium compound. In certain non-limiting embodiments, the reaction parameters are also selected to ensure that the desired final product is prepared within a commercially desirable time period and/or to result in a commercially desirable yield. In order for the reaction to proceed to the desired end product, it is necessary for most or all of the solvent to remain in liquid form to ensure a liquid phase reaction, and desirable to employ a temperature and/or pressure that is somewhat above ambient in order to expedite the reaction. In some non-limiting embodiments the reaction is desirably carried out at a temperature of at least about 60° C. (about 140° F.), and in other non-limiting embodiments the temperature is desirably at least about 80° C. (about 176° F.). In still other non-limiting embodiments the temperature is desirably at least about 100° C. (about 212° F.). Thus, it will be seen that, in the case of relatively low boiling solvents, e.g., methanol, pressures above ambient may be required in order to ensure maintenance of a liquid state during the reaction, while higher boiling solvents, e.g., glycols, may be employed at elevated temperatures without the need for greater-than-ambient pressure.

Contacting of the starting materials may be done in any way and in any type of vessel that results in formation of the desired final product, i.e., the sterically hindered quaternary ammonium compound. Because the end product includes anionic halide, laboratory or production vessels that are resistant to corrosion from halide are generally preferred. For example, reaction vessels made of glass or metal alloys, including those specifically designed to be resistant to halide corrosion such as hastalloy, may be particularly useful.

Introduction of the solvent, halohydrin and sterically hindered tertiary amine into the reaction vessel may be concurrent or in any order or combination of orders. However, those skilled in the art will keep in mind that if contact of any one starting material is significantly delayed relative to the other starting materials, side reactions, that may significantly reduce or even circumvent production of the desired final product, may occur. Simple mixing or stirring, using any conventional means known in the laboratory or production facility to maximize contact, may be employed. Production may be carried out via either batch or continuous methods.

Inclusion of a catalyst with the starting materials may also be considered. One effective type of catalyst is a second amine compound that acts as a base to heighten promotion of the ring closure of the halohydrin to form the epoxide, but which, due to either steric or electronic reasons, does not react significantly with the protonated epoxide to, itself, form a quaternary ammonium compound. For example, amines such as diazabicyclo[2.2.2]octane, diazabicyclo[5.4.0]-undecene, and mixtures thereof may, in some non-limiting embodiments, be effective for this purpose. However, it has been found that the inventive method's generalized reaction will, in some non-limiting embodiments, proceed effectively and at high yield even without a catalyst.

In many non-limiting embodiments, the method described herein results in yields, i.e., conversions, of at least about 50 percent of theoretical, and in other non-limiting embodiments the yields are at least about 80 percent. In yet other non-limiting embodiments, the yields may be at least about 90 percent of theoretical. Where smaller and structurally simpler tertiary amines are employed, yields tend to be higher than for larger and more complex tertiary amines.

The compounds thus formed are sterically hindered quaternary ammonium compounds. In some embodiments it may be desirable to employ a mixture of two or more halohydrins, in order to produce a mixture of two or more sterically hindered quaternary ammonium compounds. This approach might be selected where, for example, a single quaternary ammonium compound results in a more crystalline or otherwise less-easily-dispersed final product. In one non-limiting embodiment, a mixture of dodecyl alcohol and tetradecyl alcohol may be reacted with epichlorohydrin to produce a mixed chlorohydrin product. This mixed product is then used in the method of the present invention to result in a mixture of two sterically hindered quaternary ammonium compounds conforming to Formula 1, which may alternatively be denominated as C₂₇H₅₈ClNO₂ and C₂₉H₆₃ClNO₂. In other non-limiting embodiments the chlorine atom in these compounds may be replaced with bromine, fluorine or iodine.

The sterically hindered quaternary ammonium compounds formed by the method previously described are useful for any purpose for which quaternary ammonium compounds are known to be useful, such as surface active agents, dispersing agents, foaming agents, and corrosion inhibitors. Of particular application, however, is their notable efficacy in inhibiting formation of hydrates in susceptible fluids. In this use the quaternary ammonium compounds may be introduced into a wellbore, reservoir, or any associated or other oilfield tubulars, such as flowlines, pipelines, transfer lines, tubing, and the like, by way of equipment or methods known to those skilled in the oilfield arts. For example, the compounds may be injected by way of coiled tubing.

In amount the quaternary ammonium compounds useful in the invention for hydrate control may range, in some non-limiting embodiments, from about 0.01 to about 2.0 volume percent, based on the water or other aqueous fluid (for example, brine) that is present. In other non-limiting embodiments such may range from about 0.5 to about 1.5 volume percent. In other applications, such as when the sterically hindered quaternary ammonium compounds are used as corrosion inhibitors, surfactants, and the like, it is more typical to employ much smaller amounts, ranging from about 1 to about 5,000 ppm, and in some non-limiting embodiments amounts from about 10 to about 1,000 ppm may be desirable.

In some embodiments, the method of the invention is practiced by introducing a sterically hindered quaternary ammonium composition into a fluid passing through oil and gas production and/or exploration equipment. For the purposes of this application, the term “fluid” means production fluids which, in turn, includes formation fluids, crude oil, crude natural gas, and any other fluids encountered in the production of oil and gas. For example, production fluid may be subjected to dewatering, desalting, and the like to produce a fluid that is enriched in hydrocarbons as compared to the original production fluid. This fluid is sometime referred to in the art as “crude oil.” Also included in the term production fluids are the fluids encountered during the drilling of an oil or gas well including, but not limited to, brine, drilling muds and fluids and the like. Any fluid that present either down-hole or in any equipment associated with the production of oil and/or natural gas that my be subject to the formation of hydrates is included within the term fluid in the claims.

Similarly, the term “oil exploration and/or production equipment” means any equipment that would experience a flow of fluid during the course of drilling an oil or gas well and during the production of oil and/or gas, including the transportation of such fluids to a refinery. Included in this group are equipment such as a wellbore, flowline, pipeline, transfer line, and tubing.

The hydrate inhibitors of the application may, in some applications, be used in solution in a solvent. Solvents that may be useful include, but are not limited to alcohols having from 2 to 4 carbons, glycols having from 2 to 8 carbons, and glycol ethers having from 3 to 8 carbons. The hydrate inhibitors may also include other components such as corrosion inhibitors.

The description hereinabove is intended to be general and is not intended to be inclusive of all possible embodiments of the invention. Similarly, the examples hereinbelow are provided to be illustrative only and are not intended to define or limit the invention in any way. Those skilled in the art will be fully aware that selections of solvents and combinations of solvents, sterically hindered tertiary amines and combinations thereof, and halohydrins and combinations thereof; reaction conditions; reaction vessels; reaction protocols; hydrocarbon streams and reservoirs; and the like; may be varied within the scope of the claims appended hereto.

### EXAMPLES

### Example 1

About 4.00 g methanol; 2.00 g water; 8.02 g of a mixture of the chlorohydrin products of the reaction of epichlorohydrin with ALFOL™-1214, which is a mixture of dodecyl alcohol and tetradecyl alcohol; and 5.58 g tributylamine; were combined in a 4 ounce (about 0.1134 kg) vial. The chlorohydrin products mixture includes two compounds, of which each conformed to the general formula

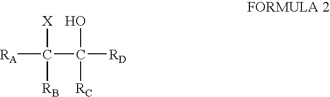

wherein X is chlorine, fluorine, bromine or iodine; and wherein R_{A}, R_{B}, and R_{C} are hydrogen and R_{D} is CH₂—O—(CH₂)ₙCH₃, and wherein n is 11 (representing one compound) and 13 (representing a second compound). The vial was loosely capped with aluminum foil and then shaken manually for about 1 minute to thoroughly mix the constituents. The vial was then placed in a stainless steel pressure bomb. The bomb was sealed and pressurized to 150 psi (about 1034 kPa) with nitrogen. It was then placed in an oven at 120° C. (about 248° F.) for 20 hours. The bomb was then removed from the oven and allowed to cool to room temperature. Then the pressure was slowly vented. The bomb was opened and the vial removed. It was found to contain a mixture of sterically hindered quaternary ammonium compounds as a yellow solution including methanol and water. These compounds were characterized as conforming to Formula 1, with one compound having as R₁ a C₁₂H₂₅ moiety, and the other having as R₁ a C₁₄H₂₉ moiety, and with R₂, R₃ and R₄ in both compounds being n-butyl. Thus, the final compounds could be alternatively characterized as C₂₇H₅₈ClNO₂ and C₂₉H₆₃ClNO₂, respectively.

### Example 2

Four 33 ml test cells, each having a sapphire glass window and containing a number of ball bearings, were selected. Into each of these test cells was charged an aqueous phase consisting of 3.0 g of an 11.0 weight percent NaCl solution and 9.0 ml of a 75 volume percent natural gas condensate from the Gulf of Mexico. A gas composition that was an 85/15 mole/mole mixture of methane and propane was also added as compressed gas to obtain a pressure of 1500 psi (about 10340 kPa). This combination of materials simulated the production composition that is frequently encountered in gas wells and particularly in subsea operations, where brine, natural gas mixtures, and liquid hydrocarbons come into contact.

The four cells were then prepared for comparative testing. Cell A included, in an amount of 1.2 volume percent based on the brine, a sterically hindered quaternary ammonium compound that is known in the art to be useful for inhibiting hydrate formation and that was commercially manufactured by a more complex method than that of the present invention. This cell was, thus, the “control.” Cell B was a “blank,” i.e., it included no gas hydrate inhibitor. Cell C and Cell D were duplicates of the same sterically hindered quaternary ammonium compound of the invention, used as the hydrate inhibitor. This quaternary ammonium compound had been previously prepared according to the method, materials and proportions used in Example 1. It was added to each of Cells C and D as a 70 volume percent by weight dilution that included 30 volume percent methanol.

The test cells' contents were pressurized initially at 1500 psi (about 10340 kPa) using compressed gas at ambient temperature, then underwent a “shock” cool-down (i.e., a rapid cool-down) to 40° F. (about 4.4° C.) in a refrigerated bath. They were then shut-in (i.e., allowed to sit without movement) for a time, followed by gentle rocking to an angle of approximately 300 from horizontal. These conditions were intended to simulate the overall conditions to which a typical production composition would likely be subjected, including extended quiescence under low temperature and high pressure, followed by rocking-like movement through pipelines and flowlines. At various points in time the test cells' contents were inspected visually to determine whether gas hydrates were forming or had formed. The ball bearings, called simply “balls” hereinbelow, simulated the effect of fluid flow in a pipeline, breaking viscosity somewhat and providing for gentle agitation of the cells' contents. The following table, designated as Table 1, shows the results of the tests.

TABLE 1

| Time from | Cell A | Cell B | | |
|---|---|---|---|---|
| start of test | (Control) | (Blank) | Cell C* | Cell D* |
| 22.5 hr; end | Clear | Clear | Clear | Clear |
| of shut-in | condensate & | condensate & | condensate & | condensate & |
| | brine; no | brine; no | brine; no | brine; no |
| | evidence of | evidence of | evidence of | evidence of |
| | hydrates | hydrates | hydrates | hydrates |
| 28.0 hr, after | Fine hydrate | Balls locked in | Fine hydrate | Fine hydrate |
| 5.5 hr of | particles; balls | a mass of | particles; balls | particles; balls |
| rocking | rock with | hydrates; | rock with | rock with |
| | ease; no | failed test | ease; no | ease; no |
| | hydrates seen | | hydrates seen | hydrates seen |
| | adhering to | | adhering to | adhering to |
| | cell's interior; | | cell's interior; | cell's interior; |
| | easy pass | | easy pass | easy pass |
| 48.75 hr, after | Fine hydrate | Balls locked in | Fine hydrate | Fine hydrate |
| 26.25 hr of | particles; balls | a mass of | particles; balls | particles; balls |
| rocking; end | rock with | hydrates; | rock with | rock with |
| of test | ease; no | failed test | ease; no | ease; no |
| | hydrates seen | | hydrates seen | hydrates seen |
| | adhering to | | adhering to | adhering to |
| | cell's interior; | | cell's interior; | cell's interior; |
| | easy pass | | easy pass | easy pass |

*includes a quaternary ammonium compound of the invention as the hydrate inhibitor

It will be seen from the table that the quaternary ammonium compound of the invention (Cells C and D) was fully as effective as the control (Cell A) at inhibiting hydrate formation, and that both of the hydrate inhibitors were much more effective than using no inhibitor at all (Cell B).

We claim:

1. A method of inhibiting hydrate formation in oil exploration and/or production equipment comprising:
detecting a reduction of flow rate for fluid passing through the oil exploration and/or production equipment;
determining whether temperature and/or pressure within the oil exploration and/or production equipment are sufficient for hydrate formation; and
if the temperature and/or pressure within the oil exploration and/or production equipment are sufficient for hydrate formation, then contacting the fluid within the oil exploration and/or production equipment with a sterically hindered quaternary ammonium composition conforming to the general formula: 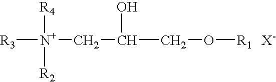
wherein the general formula includes R groups R₁, R₂, R₃, and R₄, R1 has from 1 to 20 carbon atoms; wherein R₂, R₃ and R₄ each have from 3 to 6 carbon atoms; wherein any R groups having the same number of carbon atoms may be the same or different; wherein each R group may be linear or branched; wherein each R group may have one or more carbon-to-carbon double bonds; and wherein X is a halogen selected from the group consisting of chlorine, fluorine, bromine and iodine; under conditions such that the formation of hydrates in the fluid is inhibited in comparison with a similar fluid without the sterically hindered quaternary ammonium compound.

2. The method of claim 1 wherein R₁ has 12 or 14 carbon atoms, and wherein R₂, R₃ and R₄ are each independently butyl or isobutyl moieties.

3. The method of claim 1 wherein the sterically hindered quaternary ammonium compound is prepared by a method comprising contacting a solvent having hydroxyl functionality, a sterically hindered tertiary amine, and a halohydrin, under reaction conditions sufficient to produce a sterically hindered quaternary ammonium compound.

4. The method of claim 1 wherein the solvent is selected from the group consisting of water, alcohols containing from 1 to 10 carbon atoms, glycols, and mixtures thereof.

5. The method of claim 20 wherein the sterically hindered tertiary amine is selected from the group consisting of compounds conforming to the general formula 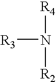
wherein R₂, R₃ and R₄ each independently have from 3 to 12 carbon atoms, may be linear or branched, and may have one or more carbon-to-carbon double bonds.

6. The method of claim 5 wherein the sterically hindered tertiary amine is selected from the group consisting of tributylamine, triisobutylamine, tripropylamine, and mixtures thereof.

7. The method of claim 1 wherein the halohydrin conforms to the general formula: 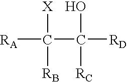
wherein X is chlorine, fluorine, bromine or iodine; and wherein R_{A}, R_{B}, R_{C} and R_{D} are each independently selected from hydrogen, hydrocarbon substituents containing from 1 to 20 carbon atoms, and heteroatoms selected from oxygen, nitrogen, phosphorus and combinations thereof.

8. The method of claim 1 wherein the reaction conditions include a temperature of at least about 60° C. (140° F.).

9. The method of claim 1 wherein the reaction conditions are selected such that the solvent is maintained in a liquid state.

10. The method of claim 1 wherein the fluid within the oil exploration and/or production equipment is selected from the group consisting of production fluid and crude oil.

11. The method of claim 10 wherein the fluid within the oil exploration and/or production equipment is brine.

12. The method of claim 1 wherein the sterically hindered quaternary ammonium composition is present in the fluid at a concentration of from about 0.01 to about 2.0 volume percent.

13. The method of claim 12 wherein the sterically hindered quaternary ammonium composition is present in the fluid at a concentration of from about 0.5 to about 1.5 volume percent.

14. The method of claim 1 wherein the oil exploration and/or production equipment is oil and gas production equipment.

15. The method of claim 14 wherein the oil and gas production equipment is a pipeline.

16. The method of claim 14 wherein the oil and gas production equipment is a wellbore, flowline, pipeline, transfer line, or tubing.

17. A method of inhibiting hydrate formation in oil exploration and/or production equipment comprising:
detecting a reduction of flow rate for fluid passing through the oil exploration and/or production equipment;
determining whether temperature and/or pressure within the oil exploration and/or production equipment are sufficient for hydrate formation; and
if the temperature and/or pressure within the oil exploration and/or production equipment are sufficient for hydrate formation, then contacting the fluid within the oil exploration and/or production equipment with a sterically hindered quaternary ammonium compound comprising contacting a solvent having hydroxyl functionality, a sterically hindered tertiary amine, and a halohydrin, under reaction conditions sufficient to produce a sterically hindered quaternary ammonium compound.

18. A hydrate inhibitor comprising a sterically hindered quaternary ammonium composition conforming to the general formula: 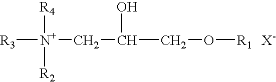
wherein the general formula includes R groups R₁, R₂, R₃, and R₄, R1 has from 1 to 20 carbon atoms; wherein R2, R3 and R₄ each have from 3 to 6 carbon atoms; wherein any R groups having the same number of carbon atoms may be the same or different; wherein each R group may be linear or branched; wherein each R group may have one or more carbon-to-carbon double bonds; and wherein X is a halogen selected from the group consisting of chlorine, fluorine, bromine and iodine.

19. The hydrate inhibitor of claim 18 further comprising a solvent.

20. The hydrate inhibitor of claim 19 further comprising a corrosion inhibitor.
